(19) United States
(12) Patent Application Publication
(10) Pub. No.: US 2015/0305925 A1
(43) Pub. Date: Oct. 29, 2015

(54) Heated Orthopedic Braces/Support/Sleeve

(71) Applicant: Brett Behrendt, Harbor Springs, MI (US)

(72) Inventor: Brett Behrendt, Harbor Springs, MI (US)

(21) Appl. No.: 14/260,311

(22) Filed: Apr. 24, 2014

(51) Int. Cl. A61F 7/00

(52) U.S. Cl. CPC A61F 7/007; A61F 2007/0086

(57) ABSTRACT

The present invention relates to a variety of battery and or AC operated heated braces/support/sleeve (Ankle, neck, knee, back, wrist, shoulder, hand, hips and elbow) that incorporates neoprene and various types of fabric material, flannel, Velcro, heating elements, battery and heat controller. The elements apply high heat to areas of muscle and tissue relieving pain temporarily and increasing blood flow to those affected areas.

### TECHNICAL FIELD OF INVENTION

The system in each therapeutic brace will help deliver moist and dry heat treatments, high temperature to relieve pain, stimulate circulation and stiffness, and relax muscle spasms and tension.

### BACKGROUND OF THE INVENTION

It is common with the medical industry that heat therapy increases blood flow and makes connective tissue more flexible. It temporarily decreases joint stiffness, pain, and muscle spasms. Heat also helps reduce inflammation and the buildup of fluid in tissues (edema). Heat therapy is used to treat inflammation (including various forms of arthritis), muscle spasm, and injuries such as sprains and strains.

The heated braces/support/sleeve of this present invention fulfills a need to have an ambulatory solution for a battery operated high heat therapy device. This allows the patient/consumer to apply heat to the affected area at anytime and at any location. Most other devices need to be plugged in to an outlet for power for high heat. Other product out on the market use gel or chemical to create heat, which works at a much lower temperature.

### SUMMARY OF THE INVENTION

In accordance with one (1) embodiment of this invention, a heated brace/support/sleeve design has heating elements embedded or stitched into the material or using a pocket design for them. The battery/temperature control device is attached related to each brace design or use a belt clip design like cell phone use. The controller is on or built on the battery or a hand held remote (wireless device) is used to control the heat temperature.

Those skilled in the art recognize that the brace/support/sleeve can incorporate a variety of designs. The brace/support/sleeve can be a design similar to braces/supports/sleeves currently known in the art. The braces/supports/sleeves will be manufactured with neoprene, fabrics, flannel or other cloth/textile fabrics. In other embodiments to this invention, the heating elements could be designed with ridged or flexible heating elements with a temperature control wireless or not and or a switch that's powered by a DC battery and or AC power.

### BRIEF DESCRIPTION OF THE DRAWINGS

FIG. 1 thru 8 is a perspective view of each brace/support/sleeve embodiment of the heated device system.

### DETAILED DESCRIPTION OF PREFERRED EMBODIMENTS

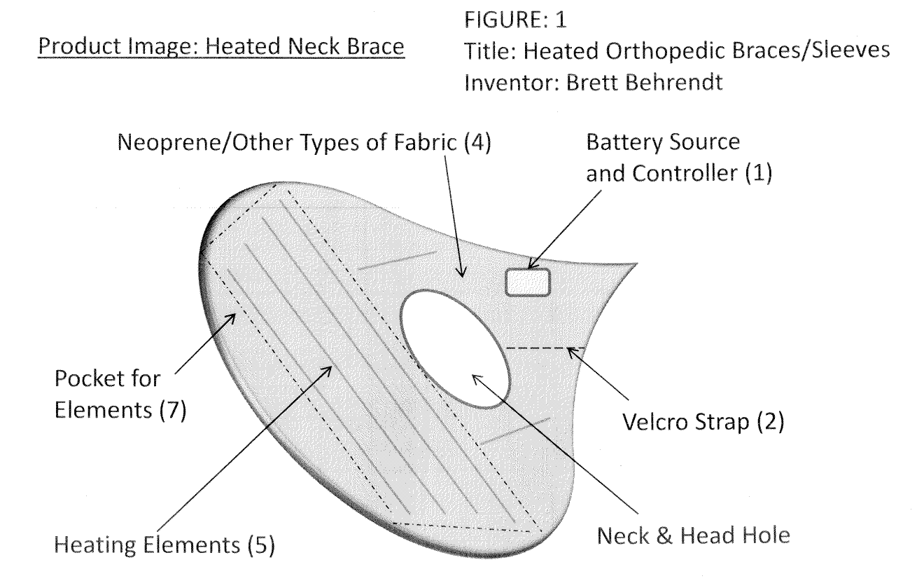

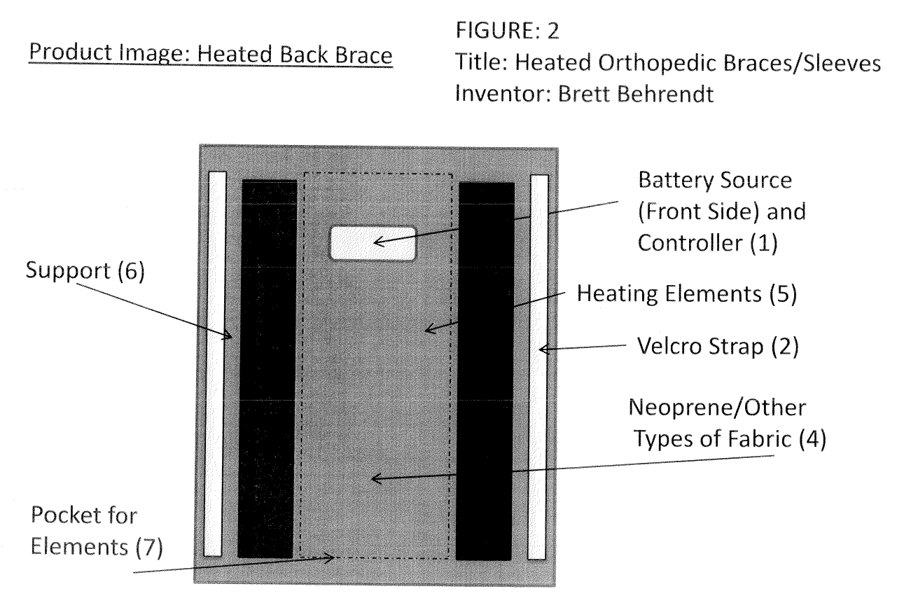

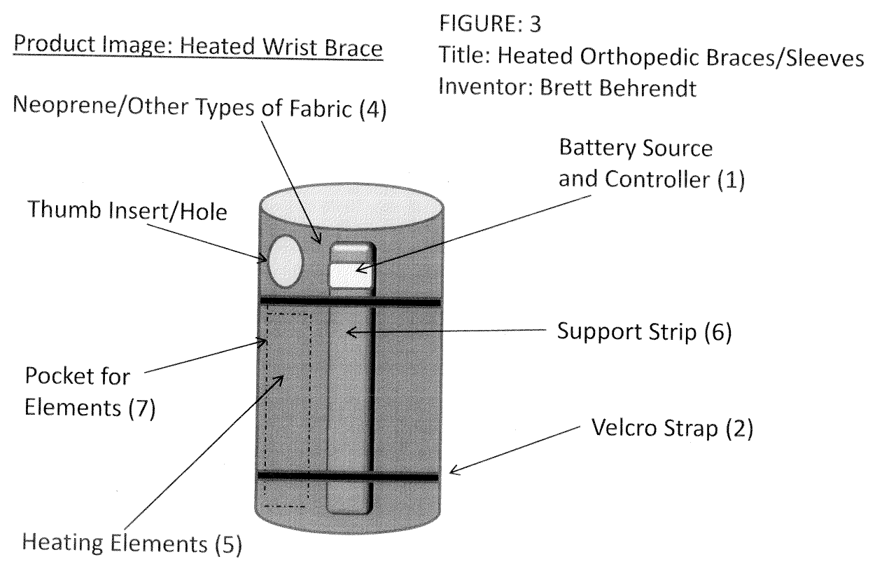

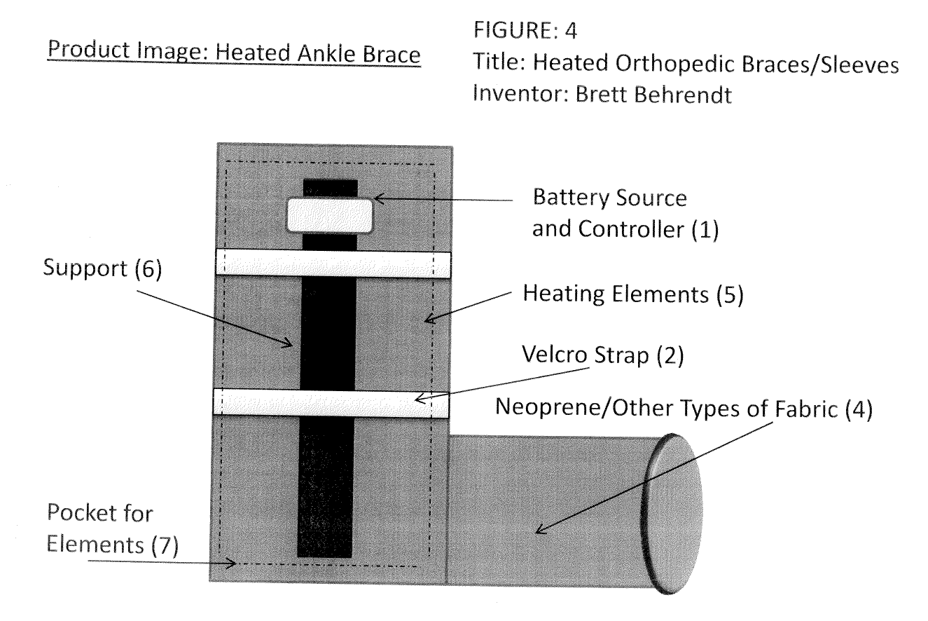

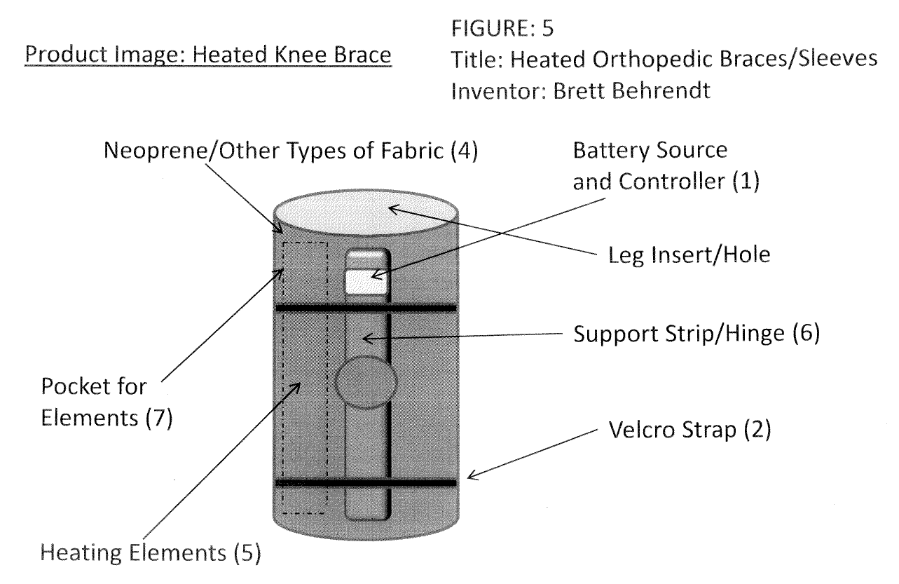

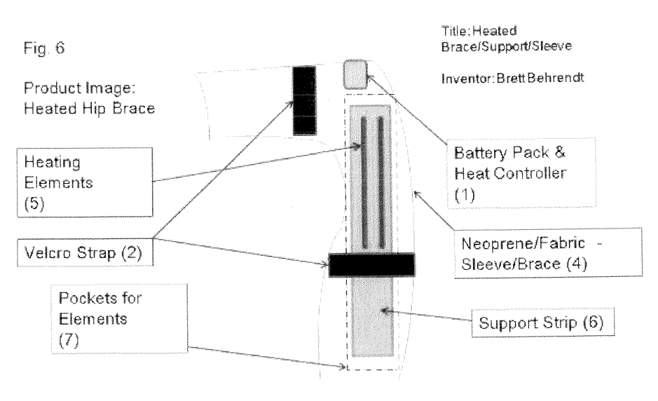

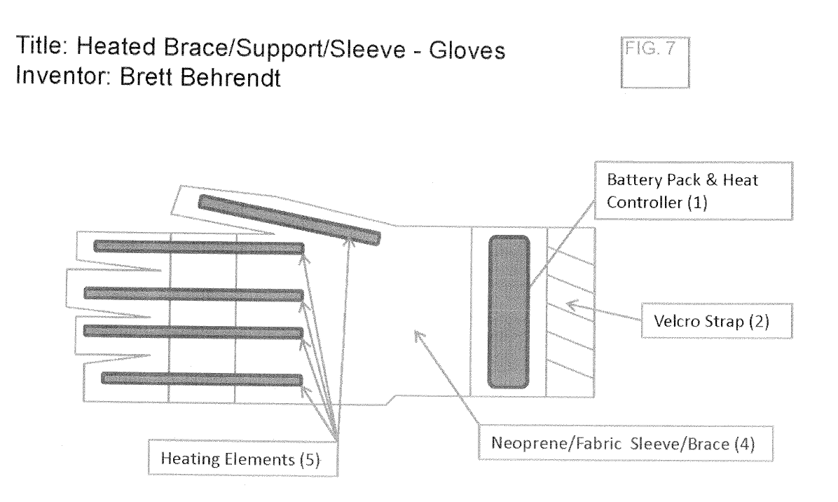

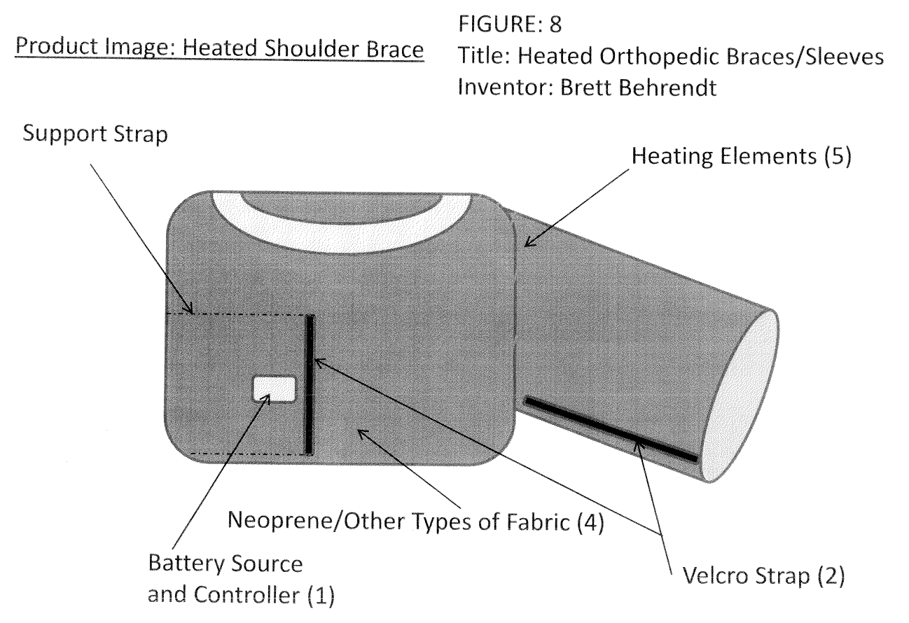

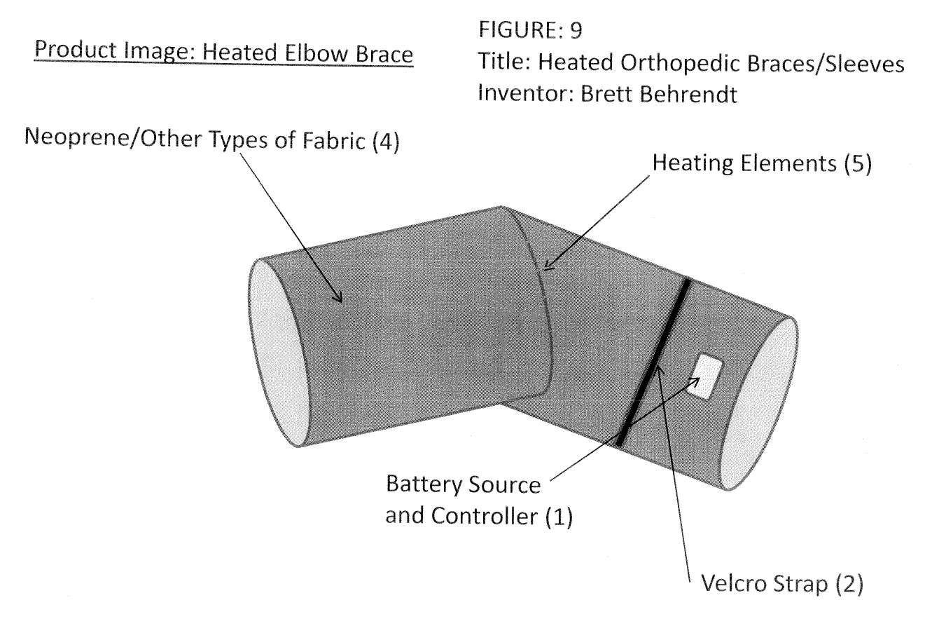

FIG. 1 show's a heated neck pad that's used to heat the neck and shoulder area this in accordance with one (1) embodiment of the present invention. The heated neck pad in Figure one (1) shows a heated neck brace includes a battery pack and heat controller (wireless or not) (1), a Velcro strap (2), neoprene or fabric material and flexible or ridged heating elements (5) and option for pockets to house heating elements (7).

In FIG. 2 show's a heated back brace used to heat the upper and or lower back, it includes a battery pack and heating temperature controller (1), Velcro strap (2), flexible or ridged heating elements (5), support strip (6) and neoprene or fabric (4) and option for pockets to house heating elements (7).

In FIG. 3 show's a heated wrist brace, it includes a battery pack and heating temperature controller (1), Velcro strap (2), flexible or ridged heating elements (5), support strip (6)and neoprene or fabric (4) and option for pockets to house heating elements (7).

In FIG. 4 show's a heated ankle brace, it includes a battery pack and heating temperature controller (1), Velcro strap (2), flexible or ridged heating elements (5), support strip (6) and neoprene or fabric (4) and option for pockets to house heating elements (7).

In FIG. 5 show's a heated knee brace, it includes a battery pack and heating temperature controller (1), Velcro strap (2), flexible or ridged heating elements (5), support strip (6) and neoprene or fabric (4) and option for pockets to house heating elements (7).

In FIG. 6 show's a heated Hip brace, it includes a battery pack and heating temperature controller (1), Velcro strap (2), flexible or ridged heating elements (5), support strip (6) and neoprene or fabric (4) and option for pockets to house heating elements (7).

In FIG. 7 show's a heated Gloves brace, it includes a battery pack and heating temperature controller (1), Velcro strap (2), flexible or ridged heating elements (5), and neoprene or fabric (4).

In FIG. 8 show's a heated Shoulder brace, it includes a battery pack and heating temperature controller (1), Velcro strap (2), flexible or ridged heating elements (5), and neoprene or fabric (4).

In FIG. 9 show's a heated Elbow brace, it includes a battery pack and heating temperature controller (1), Velcro strap (2), flexible or ridged heating elements (5), and neoprene or fabric (4).

In accordance with one (1) embodiment of this invention, a heated brace/support/sleeve design has heating elements embedded or stitched into the material and the battery/temperature control device is attached related to each brace design.

Those skilled in the art recognize that the brace/support/sleeve can incorporate a variety of designs. The brace/support/sleeve can be a design similar to braces/supports/sleeves currently known in the art. The braces/supports/sleeves will be manufactured with neoprene, fabrics, flannel or other cloth/textile fabrics. In other embodiments to this invention, the heating elements could be designed with ridged or flexible heating elements with a temperature control and or a switch that's powered by a DC battery and or AC power.

In another embodiment, the brace/support/sleeve can be design similar to other braces currently known in the art. In this embodiment various fabric and neoprene, fleece, flannel, Velcro, foam, polyester fiber, mesh, elastic and stretch knit materials could be used to manufacture the different braces/supports/sleeves for numerous parts of the body (ankle, knee, neck, wrist, hand, elbow, back, shoulder, hips).

In another embodiment, the heating elements can be designed similar to Neoprene Heaters and silicone rubber heaters but operate at a lower temperature and watt density. Neoprene is a thin, light-weight material ideally suited for flexible heaters. This heater is weather and chemical resistant with a low thermal mass, lightweight construction and the applications includes battery heaters. Other applications also include silicone rubber heaters. This invention utilizes these flexible heaters similar to companies such as Watlow, Flexelec, Exo2 and Thermo-LCC.

In another embodiment, the temperature control and or switch can be designed similar to other controllers currently know in the art. The design of the temperature controller could have settings of low, medium and high or an adjustable controller (operating environment (0-180 degrees Fahrenheit)). Controller could also have a timer or sensor shut off mode. This invention utilizes these temperature controllers similar to companies such as Watlow.

In the foregoing description, various features of the present invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie on less than all features of a single foregoing disclosed embodiment. Thus, the following claims are here by incorporated by reference herein in their entirety, with each claim standing on its own as a separate embodiment of the present invention.

It is intended that the foregoing description be only illustrative of the present invention and that the present invention be limited only by the hereinafter appended claims.

What is claimed is:

1. A heated device for braces/supports/sleeves comprising of flexible or ridged heating elements, battery pack, heating controller, support strip or hinges, neoprene, various fabrics, flannel, Velcro.

2. The heated device for braces/supports/sleeves of claim 1, wherein the battery pack powers the controller and the heating elements.

3. The heated device for braces/supports/sleeves of claim 2, wherein the heating controller is an adjustable/lock temperature control, timer, with an manual on/off switch, powered by the battery pack or an AC adapter. This device controls the temperature that is adjustable from 0-180 F degrees.

4. The heated device for braces/supports/sleeves of claim 3, wherein flexible or ridged heating elements will be used for heat therapy that increases blood flow and makes connective tissue more flexible. It temporarily decreases joint stiffness, pain, and muscle spasms. Heat also helps reduce inflammation and the buildup of fluid in tissues (edema). Heat therapy is used to treat inflammation (including various forms of arthritis), muscle spasm, and injuries such as sprains and strains.

5. The heated device for braces/supports/sleeves of claim 4, wherein Velcro straps will be used to tighten and help secure the brace/support/sleeve to the patient/customers limb or body.

6. The heated device for braces/supports/sleeves of claim 5, wherein neoprene/fabric material, flannel is known to provide the highest heat retention and insulation of any material.

7. The heated device for braces/supports/sleeves of claim 6, wherein support strips or ninge are used to provide support and relieves pressure.

8. The heated device for braces/supports of claim 7, wherein pockets (with or without Velcro closure) to hold/house the elements and/or battery to provide easy access for changing out elements and battery packs.
